(19) United States
(12) Patent Application Publication
(10) Pub. No.: US 2011/0262540 A1
(43) Pub. Date: Oct. 27, 2011

(54) Solid Pharmaceutical Composition Comprising Exemestane

(75) Inventors: Peter Henry Robert Persicaner, Boca Raton, FL (US); Rahul Sareen, Weston, FL (US)

(73) Assignee: ARROW INTERNATIONAL LIMITED, Valletta (MT)

(21) Appl. No.: 13/054,915

(22) PCT Filed: Jul. 27, 2009
(86) PCT No.: PCT/GB09/01852

§ 371 Date: Jul. 13, 2011

(30) Foreign Application Priority Data
Jul. 25, 2008 (GB) 0813628.5

(51) Int. Cl. A61K 31/569; A61P 35/00; A61K 9/28; C07J 1/00; A61K 9/14

(52) U.S. Cl. 424/474; 552/639; 424/400; 514/177; 514/171

(57) ABSTRACT

A solid pharmaceutical composition of exemestane, in particular for oral use and treatment of cancer, comprises micronized exemestane and anti-oxidant in a film-coated tablet.

### FIELD

The present invention relates to solid pharmaceutical compositions of exemestane, and in particular to compositions suitable for oral use, uses thereof in the treatment of cancer, and processes for their preparation.

### BACKGROUND

Exemestane, which has the chemical name 6-methylenandrosta-1,4-diene-3,17-dione and is marketed as Aromasin®, is an irreversible, steroidal aromatase inactivator, which is structurally related to the natural substrate androstenedione. It acts as a false substrate for the aromatase enzyme, and is processed to an intermediate that binds irreversibly to the active site of the enzyme causing its inactivation. Exemestane significantly lowers circulating estrogen concentrations in postmenopausal women and it is therefore useful for the treatment of a variety of estrogen-dependent tumours.

U.S. Pat. No. 4,808,616 and United Kingdom Patent 2 177 700 relate to a series of 6-alkylidenandrosta-1,4-diene-3,17-dione derivatives, which encompass exemestane, and provides methods for their preparation and use. European Patent 0 253 591 relates to a series of 6- or 7-alkylidenandrosta-1,4-diene-3,17-dione derivatives, which encompass exemestane, and provides methods for their preparation and use.

International Patent Application WO 2002/20000 relates to methodologies for the treatment, prevention and inhibition of breast cancer by administering exemestane in combination with raloxifene, a nonsteriodal anti-estrogen. International Patent Application WO 2007/045027 related to compositions, methods and kits for the amelioration of side effects associated with aromatase inhibitor treatment by administering an aromatase inhibitor in combination with an androgenic agent.

Exemestane is practically insoluble in water, and it is therefore difficult to make formulations suitable for oral use due to the poor rate and extent of dissolution in aqueous media (including gastrointestinal fluids), which results in low absorption into systemic circulation. In order to overcome this problem, exemestane has been formulated with an inert hydrophilic carrier and a surfactant such as polyethylene glycol which aids dissolution of the drug.

In an experimental study, Martini et al. (Int. J. Pharm. 75:141-146, 1991) found that high-energy cogrinding of micronized exemestane with crosslinked polyvinylpyrrolidone increased the solubility, dispersibility and dissolution of exemestane in a manner that was solely attributable to the physico-chemical changes to exemestane caused by the extensive co-processing.

There is, however, an additional problem associated with exemestane, in that the compound is readily susceptible to oxidation, which results in poor chemical stability, an issue which has detrimental effects on the potential shelf life of the product. Traditional means for enhancing stability of pharmaceutical compositions involves the use of antioxidants. Unfortunately, there is an incompatibility between the more preferred antioxidants and the polyethylene glycol used in exemestane formulations. The marketed product Aromasin® uses a sugar coating on the tablet instead of an antioxidant in the formulation to enhance stability. It is not clear whether such a coating is suitable for alternative formulations. Moreover, sugar coating on tablets is not always applicable or desirable.

The formation of chemical complexes between exemestane and the oligosaccharide, beta-cyclodextrin, has been said to increase the stability of exemestane. An experimental study (Torricelli et al., Int. J. Pharm. 75:147-153, 1991) and International Patent applications WO 2002/020020 and WO 2002/072106 relate to the use of kneaded systems to produce such exemestane complexed to beta-cyclodextrin.

However, an experimental study by Torricelli et at. (Int. J. Pharm. 71:19-24) found that cogrinding of micronized exemestane with beta-cyclodextrin did not aid the stability of exemestane.

There exists a need, therefore, for pharmaceutical compositions of exemestane, particularly solid pharmaceutical compositions for oral use, which address these issues. In particular, compositions of exemestane should be stable and provide a long shelf life. They should provide a good dissolution release profile and good solubilisation of the drug in aqueous media, particularly in gastrointestinal fluids. The compositions should ideally be easy to handle and be readily formulated into desired dosage forms, such as tablets or capsules, as required.

International patent application, WO 2005/074890 refers to semisolid matrix formulations comprising oxidation-susceptible and poorly water soluble drugs, such as exemestane, which are formulated using the hydrophilic carrier polyethylene glycol and water-soluble Vitamin E derivatives as antioxidant.

International patent application, WO 2001/087266 refers to stabilised aqueous suspensions for parenteral use consisting of micronized exemestane and methionine, which is said to stabilise the suspension by controlling the pH of the aqueous solution.

International patent application, WO 2001/087262 refers to stabilised aqueous suspensions for parenteral use consisting of micronized exemestane and pH controlling effective concentration of a polyvinylpyrrolidone, which is said to stabilise the suspension.

It is an objective of the present invention to provide solid pharmaceutical compositions of exemestane, which address these disadvantages in order to provide alternative or equivalent products.

### Invention

It has been surprisingly found that solid compositions of exemestane that have a good dissolution release profile and good drug solubilisation can be prepared without using the hydrophilic carrier, polyethylene glycol (PEG) 6000. Accordingly, the present invention permits the use of the more common and preferred antioxidants in order to stabilise the solid compositions. Additionally, a wider variety of coatings can be used in tablets according to the present invention.

Accordingly, in a first aspect, the present invention provides a solid pharmaceutical composition comprising micronized exemestane. In particular, the pharmaceutical composition of the invention is suitable for oral administration, such as in the form of a tablet or capsule, in particular an immediate release tablet dosage form.

Preferably, the amount of exemestane in the composition is in the range of from about 1% to about 80% by weight, more preferably from about 5% to about 60% by weight, yet more preferably from about 10% to about 50% by weight, yet more preferably from about 20% to about 40% by weight, and most preferably from about 25% to about 35% by weight.

Preferably, the exemestane has a mean particle size of less than about 40 μm, more preferably less than about 20 μm, yet more preferably less than about 15 μm, and most preferably less than about 10 μm. Preferably, 90% of the exemestane has a particle size of less than about 40 μm and 50% less than about 20 μm as determined by the Malvern method. More preferably, 90% of the exemestane has a particle size of less than about 20 μm and 50% less than about 10 μm. By mean size is meant mass median diameter, determined by light scattering methods, for example using a Malvern Mastersizer® or similar method.

Preferably, the composition further comprises a surface active agent (surfactant), such as sodium lauryl sulfate and polysorbate 80. Polysorbate 80 is the preferred surfactant for use in the pharmaceutical compositions of the invention.

Preferably, the amount of surfactant is in the range of from about 0.1% to about 5% by weight, preferably from about 0.25% to about 3% by weight, more preferably from about 0.5% to about 2% by weight, and most preferably about 1.5% of the composition.

Preferably, the composition further comprises an antioxidant. Preferred antioxidants for use in the compositions of the present invention are selected from butylated hydroxytoluene (BHT), butylated hydroxyanisole (BHA), ascorbic acid, alpha tocopherol, ascorbyl palmitate, propyl gallate, citric acid, isoascorbic acid, sodium metabisulfite, sodium sulfite, sodium bisulfite, sodium ascorbate, hydroquinone, and Vitamin E TPGS, and combinations thereof. Preferably, the antioxidant is butylated hydroxyanisole (BHA), either alone or in combination with butylated hydroxytoluene (BHT).

The amount of antioxidant is chosen to be effective to protect the active ingredient from oxidation. Suitable levels are in the range of from about 0.01% to about 2% by weight, more preferably from about 0.05% to about 1.5% by weight, yet more preferably from about 0.1% to about 1% by weight, yet more preferably from about 0.2% to about 0.6% by weight, and most preferably from about 0.3% to about 0.4% by weight.

Preferably, the composition is substantially free of cyclodextrins.

Preferably, the composition further comprises a binder. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose (HPC) and hydroxypropyl methylcellulose, and combinations thereof. Preferably, the binder is hydroxypropyl cellulose (HPC) of low molecular weight grade. Most preferably the hydroxypropyl cellulose is Klucel EF®, which is provided by Hercules GmbH (Aqualon Group).

Preferably, the amount of binder in the composition is in the range of from about 1% to about 20% by weight, more preferably from about 1% to about 15% by weight, yet more preferably from about 2% to about 12% by weight, yet more preferably from about 3% to about 10% by weight, yet more preferably from about 4% to about 8% by weight, and most preferably from about 5% to about 7% by weight.

The composition may further comprise a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Preferred disintegrants for use in the pharmaceutical compositions of the invention include crospovidone and sodium starch glycolate or a combination thereof.

Preferably, the amount of disintegrant is in the range of from about 0.1% to about 20% by weight, more preferably from about 0.5% to about 15% by weight, yet more preferably from about 1% to about 10% by weight, yet more preferably from about 2% to about 6% by weight, and most preferably from about 3% to about 4% by weight.

The solid pharmaceutical compositions of the invention also generally contain lubricants such as magnesium stearate, calcium stearate, zinc stearate, sodium stearyl fumarate, and mixtures of magnesium stearate with sodium lauryl sulphate. Lubricants preferably constitute from about 0.25% to about 10% by weight, most preferably from about 0.5% to about 3% by weight, of the tablet. Magnesium stearate is the preferred lubricant for use in the pharmaceutical compositions of the invention.

Methods for the preparation of pharmaceutical compositions of the present invention will be readily apparent to those skilled in the art and may be found, for example, in Remington's Pharmaceutical Sciences, 19th Edition (Mack Publishing Company, 1995). In particular the pharmaceutical compositions of the invention may be prepared by wet granulation using water as a granulation aid, wet granulation using non-aqueous (alcohol) as a granulation aid, wet granulation using hydro alcoholic granulation aid, or dry granulation or compaction or slugging.

The pharmaceutical compositions of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules (hard and soft gelatin) containing particulates, or powders, lozenges, chewable tablets, dry suspensions, suspensions or oral solutions, multi- and nano-particulates, gels, solid solution, liposome, orally disintegrating or lyophilized tablets, films and ovules.

The pharmaceutical compositions of the invention may also be fast-dissolving, fast-disintegrating dosage forms such as those described in Expert Opinion in Therapeutic Patents, 11 (6), 981-986, by Liang and Chen (2001).

The solid pharmaceutical compositions of the invention may also contain diluents/fillers, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Mannitol is the preferred diluent/filler for use in the pharmaceutical compositions of the invention. Preferably the amount of diluent/filler is from about 0% to about 85% by weight, more preferably from about 40% to about 85%, and most preferably from about 25% to about 65%.

The solid pharmaceutical compositions of the invention may also optionally comprise glidants such as silicon dioxide and talc. When present, glidants may comprise from about 0.2% to about 5% by weight, preferably from about 0.5% to about 1% by weight of the composition. Colloidal silicon dioxide is the preferred glidant for use in the pharmaceutical compositions of the invention.

Tablets may also be coated, preferably film-coated and polyvinyl based polymers such as Opadry™ AMB is the preferred film-coating according to the present invention.

For example, tablets may contain from about 1% to about 80% by weight exemestane having a mean particle size of less than about 40 μm, from about 0.1% to about 5% by weight surfactant, from about 0.01% to about 2% by weight antioxidant, from about 1% to about 20% by weight binder, from about 0.1% to about 20% by weight disintegrant, from about 0.25% to about 10% by weight lubricant, and from about 0% to about 85% by weight diluent/filler.

More preferably, tablets may contain from about 20% to about 40% by weight exemestane having a mean particle size of less than about 20 μm, from about 0.25% to about 3% by weight surfactant, from about 0.1% to about 1% by weight antioxidant, from about 3% to about 10% by weight binder, from about 1% to about 10% by weight disintegrant, from about 0.5% to about 3% by weight lubricant, and from about 40% to about 85% by weight diluent/filler.

Yet more preferably, tablets may contain from about 25% to about 35% by weight exemestane having a mean particle size of less than about 20 μm, from about 0.5% to about 2% by weight surfactant, from about 0.3% to about 0.4% by weight antioxidant, from about 5% to about 7% by weight binder, from about 3% to about 4% by weight disintegrant, from about 0.5% to about 3% by weight lubricant, and from about 40% to about 85% by weight diluent/filler.

In a preferred embodiment, tablets may contain from about 25% to about 35% by weight exemestane having a mean particle size of less than about 20 μm, from about 0.5% to about 2% by weight polysorbate 80, from about 0.3% to about 0.4% by weight butylated hydroxyanisole (BHA), either alone or in combination with butylated hydroxytoluene (BHT), preferably in about a 1:1 ratio, from about 5% to about 7% by weight hydroxypropyl cellulose (HPC), from about 3% to about 4% by weight crospovidone or sodium starch glycolate or a combination thereof, from about 0.5% to about 3% by weight magnesium stearate, and from 40% to about 85% by weight mannitol. Preferably the tablet is coated with Opadry™ AMB.

Further preferred embodiments of the invention are as detailed in the Experimental section.

The drug substance, exemestane, may be micronized using standard techniques known to those skilled in the art, such as milling and grinding.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Other possible ingredients include colourants, flavourings and flavour enhancers, preservatives, such as methyl paraben, salivary stimulating agents, cooling agents, co-solvents (including oils), emollients, bulking agents, anti-foaming agents, such as simethicone, and taste-masking agents.

In another aspect the invention provides a method for the manufacture of a tablet comprising the solid pharmaceutical composition according to any of the above aspects of the present invention, wherein said method comprises the steps of:
(a) preparing a powder by blending micronized exemestane with the filler and part of the disintegrant,
(b) preparing a solution containing the binding agent, surfactant and antioxidant in absolute ethanol,
(c) granulating the powder blend from step (a) with the solution from step (b),
(d) drying the granules obtained in step (c),
(e) screening and milling the dried granules obtained in step (d),
(f) blending the granules from step (e) with the rest of the disintegrant, the glidant and lubricant,
(g) compressing the granules into tablets, and
(h) film coating the tablets.

The invention additionally provides a composition as described herein for use in chemoprevention or the treatment of advanced hormone-dependent breast, cervical, pancreatic, endometrial and ovarian cancers, prostatic hypertrophy and prostatic hyperplasia.

The invention additionally provides a method for chemopreventing or for the treatment of advanced hormone-dependent breast, cervical, pancreatic, endometrial and ovarian cancers, prostatic hypertrophy and prostatic hyperplasia comprising administering to a patient in need thereof a composition as described herein.

The invention is now illustrated in a specific embodiment in the following example.

### EXAMPLE 1

### Exemestane Tablets Quantitative Formulation

TABLE 1

| Item | | | mg/ |
|---|---|---|---|
| No. | INGREDIENTS | FUNCTION | tablet |
| 1 | Exemestane | Active | 25.00 |
| 2 | Mannitol Powdered | Filler | 44.28 |
| 3 | Crospovidone | Disintegrant | 0.80 |
| 4 | Sodium Starch Glycolate | Disintegrant | 0.80 |
| 5 | Hydroxypropyl Cellulose | Binder | 4.80 |
| 6 | Polysorbate 80 | Surfactant/Wetting agent | 1.20 |
| 7 | Butylated | Anti oxidant | 0.16 |
| | Hydroxyanisole (BHA) | | |
| 8 | Butylated | Anti oxidant | 0.16 |
| | Hydroxytoluene (BHT) | | |
| 9 | Ethanol Absolute | Granulation Aid | qs |
| 10 | Sodium Starch Glycolate | Disintegrant | 0.80 |
| 11 | Crospovidone | Disintegrant | 0.80 |
| 12 | Colloidal Silicon Dioxide | Glidant | 0.80 |
| 13 | Magnesium Stearate | Lubricant | 0.40 |
| | Sub Total | — | 80.00 |
| 14 | Opadry ® AMB OY-B- | Coating Polymer/Atmospheric | 4.80 |
| | 28920 White* | Moisture Barrier | |
| 15 | Purified Water | Coating Aid | qs |
| | Total | | 84.80 |

*Components consist of Polyvinyl alcohol-part. Hydrolysed, Titanium Dioxide, Talc, Lecithin (Soya) & Xanthan Gum.

Tablets were prepared as follows using the quantities of active ingredients and excipients according to Table 1.

Micronized exemestane having a mean particle size of less than 10 μm was prepared by jet-milling.

Micronized exemestane, mannitol (160 C), a part of crospovidone and sodium starch glycollate were sifted, blended and heated in a top spray fluid bed processor. The blend was then granulated with a solution of hydroxypropyl cellulose (Klucel® EF), polysorbate 80, butylated hydroxyanisole (BHA) and butylated hydroxytoluene (BHT) in absolute ethanol. The granules were then dried in the same fluid bed processor. The dried granules were screened and milled using a standard apparatus (e.g. Comil® available from Quadro®) to achieve size uniformity. The granules had a mean particle size of about 80 to 250 μm.

The milled granules were blended with colloidal silicon dioxide and remaining crospovidone and sodium starch glycolate followed by magnesium stearate in a V blender. The final blend was compressed into tablets which were film coated with a dispersion of Opadry™ AMB OY-B-28920 White in purified water.

### EXAMPLE 2

### Dissolution Profile

The tablets of Example 1 have exhibited a similar dissolution profile to that of Aromasin® when tested by FDA's recommended methodology and in-house methodology. The results are shown in Table 1 and Table 2.

TABLE 1
Dissolution as per FDA's methodology:
(http://www.accessdata.fda.gov/scripts/cder/dissolution/
dsp_SearchResults_Dissolutions.cfm)

| Product | Aromasin | Example 1 |
|---|---|---|
| Batch No. | 4MYG74 | FDH0956A |
| Volume | 900 ml | 900 ml |
| USP I or II | I | I |
| Medium | 0.5%(w/v) | 0.5%(w/v) |
| | SLS Solution | SLS Solution |
| RPM | 100 | 100 |
| 0 min | 0 | 0 |
| 10 min | 104% | 92% |
| 20 min | 105% | 99% |
| 30 min | 105% | 99% |
| 45 min | 105% | 99% |

TABLE 2
Dissolution as per In-house methodology:

| Product | Aromasin | Example 1 |
|---|---|---|
| Batch No. | 4MYG61 | FDH0956A |
| Volume | 900 ml | 900 ml |
| Medium | 0.1M HCl | 0.1M HCl |
| USP I or II | II | II |
| RPM | 50 | 50 |
| 0 min | 0 | 0 |
| 5 min | 35% | 32% |
| 10 min | 49% | 44% |
| 20 min | 62% | 59% |
| 30 min | 69% | 66% |
| 45 min | 75% | 73% |
| 60 min | 78% | 77% |

The invention thus provides stable solid compositions comprising exemestane.

1. A solid pharmaceutical composition comprising micronized exemestane.

2. A composition according to claim 1 wherein the composition is for oral administration.

3. A composition according to claim 2 which is in the form of either a tablet or a capsule.

4. A composition according to any one of claims 1 to 3 wherein the amount of exemestane is in the range of from about 1% to about 80% by weight.

5. A composition according to claim 4 wherein the amount of exemestane is in the range of from about 10% to about 50% by weight.

6. A composition according to claim 5 wherein the amount of exemestane is in the range of from about 25% to about 35% by weight.

7. A composition according to any one of claims 1 to 6 wherein the exemestane has a mean particle size of less than about 40 μm.

8. A composition according to claim 7 wherein the exemestane has a mean particle size of less than about 20 μm.

9. A composition according to claim 7 wherein the exemestane has a mean particle size of less than about 10 μm.

10. A composition according to any one of claims 1 to 9 wherein the composition further comprises a surfactant.

11. A composition according to claim 10 wherein the surfactant is polysorbate 80.

12. A composition according to either claim 10 or claim 11 wherein the amount of surfactant is in the range of from about 0.1% to about 5% by weight.

13. A composition according to claim 12 wherein the amount of surfactant is in the range of from about 0.25% to about 3% by weight.

14. A composition according to any one of claims 1 to 13 wherein the composition further comprises an antioxidant.

15. A composition according to claim 14 wherein the antioxidant is selected from butylated hydroxytoluene (BHT), butylated hydroxyanisole (BHA), ascorbic acid, alpha tocopherol, ascorbyl palmitate, propyl gallate, citric acid, isoascorbic acid, sodium metabisulfite, sodium sulfite, sodium bisulfite, sodium ascorbate, hydroquinone, and Vitamin E TPGS, and combinations thereof.

16. A composition according to claim 15 wherein the antioxidant is butylated hydroxyanisole (BHA), either alone or in combination with butylated hydroxytoluene (BHT).

17. A composition according to any one of claims 14 to 16 wherein the amount of antioxidant is in the range of from about 0.01% to about 2% by weight.

18. A composition according to claim 17 wherein the amount of antioxidant is in the range of from about 0.3% to about 0.4% by weight.

19. A composition according to any one of claims 1 to 18 wherein the composition further comprises a binder.

20. A composition according to claim 19 wherein the binder is selected from microcrystalline cellulose, gelatin, sugars, polyethylene glycol, natural and synthetic gums, polyvinylpyrrolidone, pregelatinised starch, hydroxypropyl cellulose (HPC) and hydroxypropyl methylcellulose, and combinations thereof.

21. A composition according to claim 20 wherein the binder is hydroxypropyl cellulose (HPC).

22. A composition according to any one of claims 19 to 21 wherein the amount of binder is in the range of from about 1% to about 20% by weight.

23. A composition according to claim 22 wherein the amount of binder is in the range of from about 3% to about 10% by weight.

24. A composition according to claim 23 wherein the amount of binder is in the range of from about 5% to about 7% by weight.

25. A composition according to any one of claims 1 to 24 wherein the composition further comprises a disintegrant.

26. A composition according to claim 25 wherein the disintegrant is crospovidone or sodium starch glycolate or a combination thereof.

27. A composition according to any one of claims 1 to 26 wherein the composition further comprises a lubricant.

28. A composition according to claim 27 wherein the lubricant is magnesium stearate.

29. A composition according to any one of claims 1 to 28 wherein the composition further comprises a filler.

30. A composition according to claim 29 wherein the filler is mannitol.

31. A composition according to any one of claims 1 to 30 which is a coated tablet.

32. A composition according to claim 31 which is a film-coated tablet.

33. A composition according to claim 1 wherein the composition is in the form of a tablet comprising from about 25% to about 35% by weight exemestane having a mean particle size of less than about 10 μm, from about 0.5% to about 2% by weight polysorbate 80, from about 0.3% to about 0.4% by weight butylated hydroxyanisole (BHA) in combination with butylated hydroxytoluene (BHT) in about a 1:1 ratio.

34. A tablet according to claim 33 which further comprises from about 5% to about 7% by weight hydroxypropyl cellulose (HPC), from about 3% to about 4% by weight crospovidone in combination with sodium starch glycolate in about a 1:1 ratio, from about 0.5% to about 3% by weight magnesium stearate, and from about 40% to about 85% by weight mannitol, said tablet having a coating of Opadry™ AMB.

35. A composition according to any one of claims 1 to 34 for use in chemoprevention or treatment of advanced hormone-dependent breast, cervical, pancreatic, endometrial and ovarian cancers, prostatic hypertrophy and prostatic hyperplasia.

36. A method for chemopreventing or for treatment of advanced hormone-dependent breast, cervical, pancreatic, endometrial and ovarian cancers, prostatic hypertrophy and prostatic hyperplasia comprising administering to a patient in need thereof a composition according to any one of claims 1 to 34.

37. A method for the manufacture of a tablet comprising the solid pharmaceutical composition according to any of the claims 1 to 34, wherein said method comprises the steps of:
(a) preparing a powder by blending micronized exemestane with part of the filler and part of the disintegrant,
(b) preparing a solution containing the binding agent, surfactant and antioxidant in absolute ethanol,
(c) granulating the powder blend from step (a) with the solution from step (b),
(d) drying the granules obtained in step (c),
(e) screening and milling the dried granules obtained in step (d),
(f) blending the granules from step (e) with the rest of the disintegrant, the glidant and lubricant,
(g) compressing the granules into tablets, and
(h) film coating the tablets.

38. A solid pharmaceutical composition comprising micronized exemestane substantially as hereinbefore described.
